(19) United States
(12) Patent Application Publication
(10) Pub. No.: US 2007/0208431 A1
(43) Pub. Date: Sep. 6, 2007

(54) Swing Phase Control Device

(76) Inventors: Hardy Bisinger, Rosenfeld (DE); Volker Marek, VS-Schwenningen (DE); Gerhard Fitzlaff, VS-Schwenningen (DE)

Correspondence Address: BARLEY SNYDER, LLC, 1000 WESTLAKES DRIVE, SUITE 275, BERWYN, PA, 19312, US

(21) Appl. No.: 11/569,240

(22) PCT Filed: Apr. 27, 2005
(86) PCT No.: PCT/EP05/04520

§ 371 Date: Nov. 16, 2006

(30) Foreign Application Priority Data
May 18, 2004 (DE) 20 2004 008 014.1

(51) Int. Cl. A61F 2/64

(52) U.S. Cl. 623/39

(57) ABSTRACT

A swing phase control device for an artificial knee joint is disclosed. The swing phase control device has a piston-cylinder device having a piston having a first side and a second side opposite the first side, a first chamber associated with the first side of the piston; and a second chamber associated with the second side of the piston. The swing phase control device also has a first throttle for throttling discharge of a fluid from the first chamber and configured to be closed by a first device when a predetermined pressure is reached or exceeded within the first chamber; and a second throttle for throttling discharge of a fluid from the second chamber and configured to be closed by a second device when a predetermined pressure is reached or exceeded within the second chamber.

The present invention relates to a swing phase control device for an artificial knee joint with a piston-cylinder device.

Such a piston-cylinder device is known from EP 0 615 430 B1, for example. The swing phase control device described there comprises a first chamber on one side of the piston and a second chamber on the opposite side of the piston wherein the two chambers are connectable to each other via a first throttle. A second throttle is provided for connecting the second chamber to a fluid reservoir wherein a device is constructed such that it closes the second throttle when a predetermined pressure is reached or exceeded in the second chamber.

This swing phase control achieves the effect that retracting of the piston of the piston-cylinder device and thus bending of the artificial knee joint can be controlled by the second throttle and that the throttling takes place dependent on the retraction speed of the piston. Due to this, different controlling can be realized for the bending of the knee joint and thus a more natural course of motion is made possible.

If the knee joint shall be extended again, the person using the artificial knee joint has to swing the lower leg frontward again and bring it straight with the thigh. In doing so, with the known swing phase control, a predetermined throttling effect occurs in which the throttling takes place by means of the first throttle and does not depend on the speed of extending of the artificial knee joint.

However, since in a natural course of motion the process of extending takes place dependent on the step speed as well, such throttling leads to an unnatural course of motion. Indeed, the first throttle is adjustable and thus can be adapted to the respective walking style, however, it does not comprise a dynamic adaptation to different courses of motion of the person. Thus, the first throttle acts in an optimum way either for slow or for fast movements only.

It is the object of the invention to create a swing phase control of the kind described above which allows a natural course of motion and which in particular is suited for extension motions of the artificial knee joint with different speeds.

The object is solved by a swing phase control according to claim 1. Further developments of the invention are specified in the dependent claims.

The swing phase control according to the invention has the particular advantage that it provides, for the motion of extension too, a throttling which is dependent on the speed of motion and which is adjustable independent from the throttling of the bending motion. Thus, a course of motion is allowed which looks more natural. If a fast motion of extension takes place, the piston moving out from the cylinder very rapidly reduces the volume of the first chamber and compresses the fluid contained therein. When the pressure exceeds a predetermined value, the first throttle becomes blocked and damping due to the compressed fluid occurs.

Thus, with the swing phase control according to the present invention natural walking full of verve is possible with the prosthesis.

Further features and advantages of the invention will become apparent from the description of embodiment examples with reference to the enclosed drawings. In the figures:

FIG. 1 shows a side view of a swing phase control device of an embodiment of the present invention;

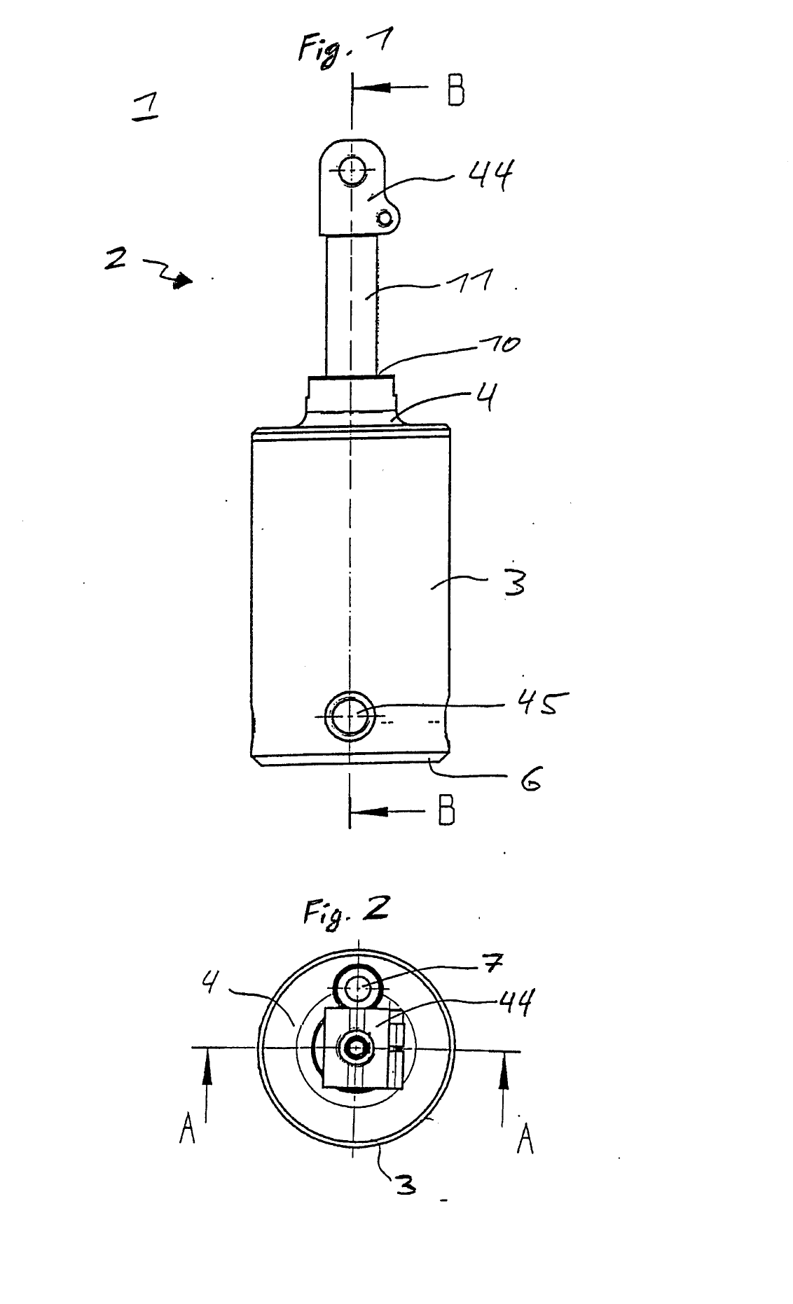

FIG. 2 shows a top view onto the swing phase control device of FIG. 1;

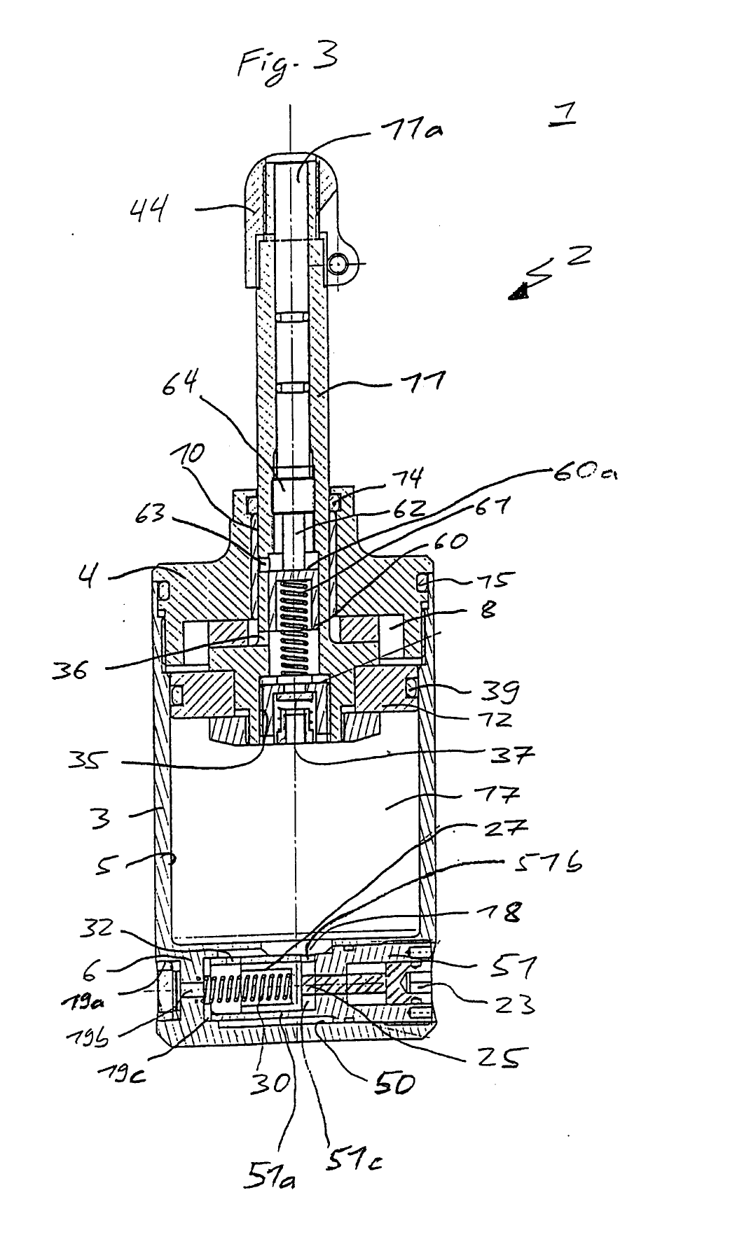

FIG. 3 shows a section along line A-A in FIG. 2;

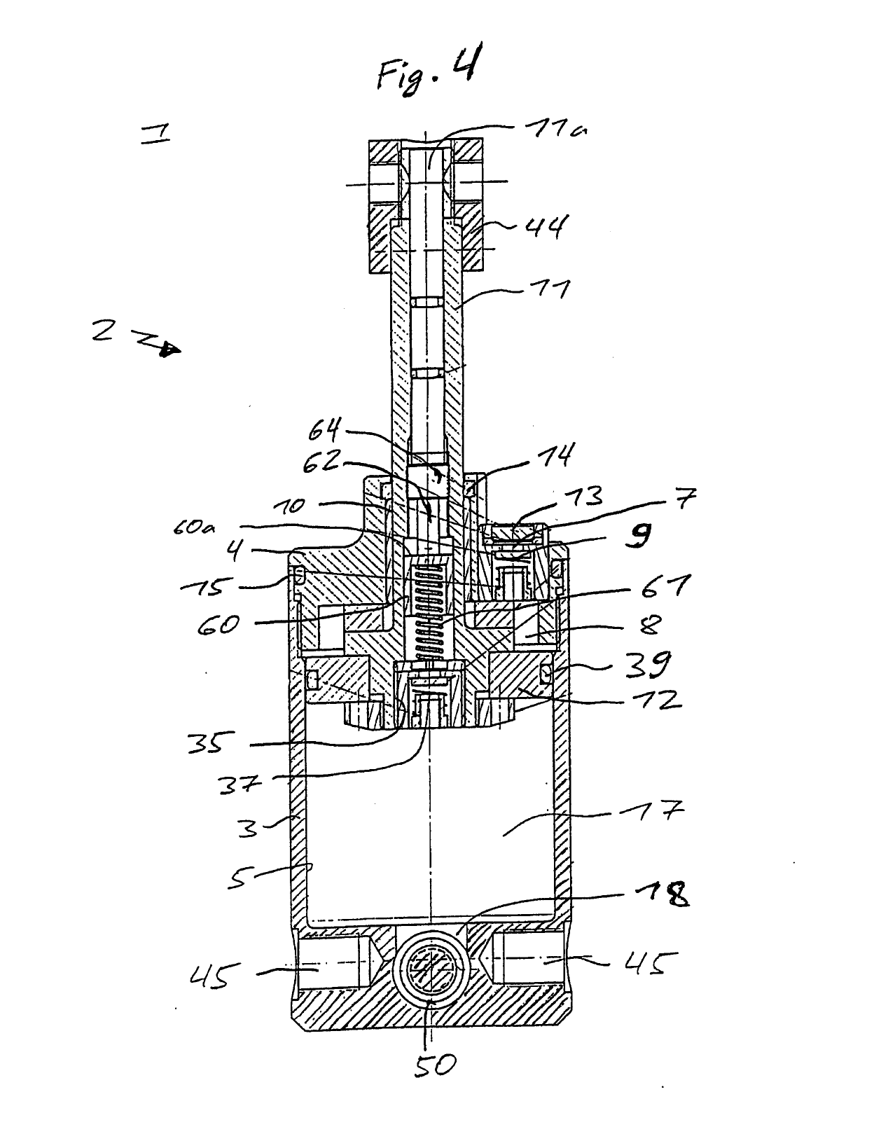

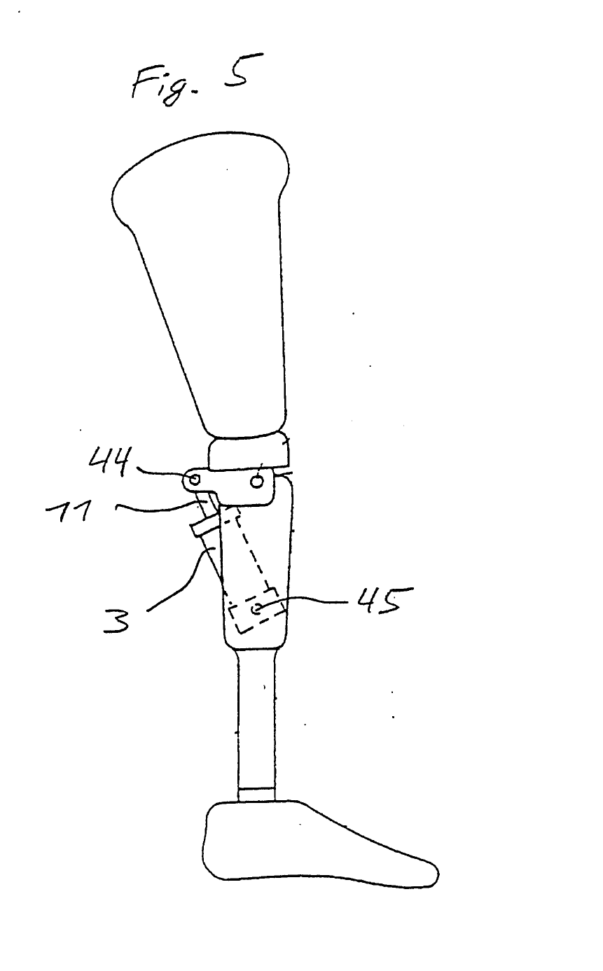

FIG. 4 shows a section along line B-B in Fig, 1;

FIG. 5 shows an illustration showing the use of the swing phase control device in an artificial knee of a leg prosthesis;

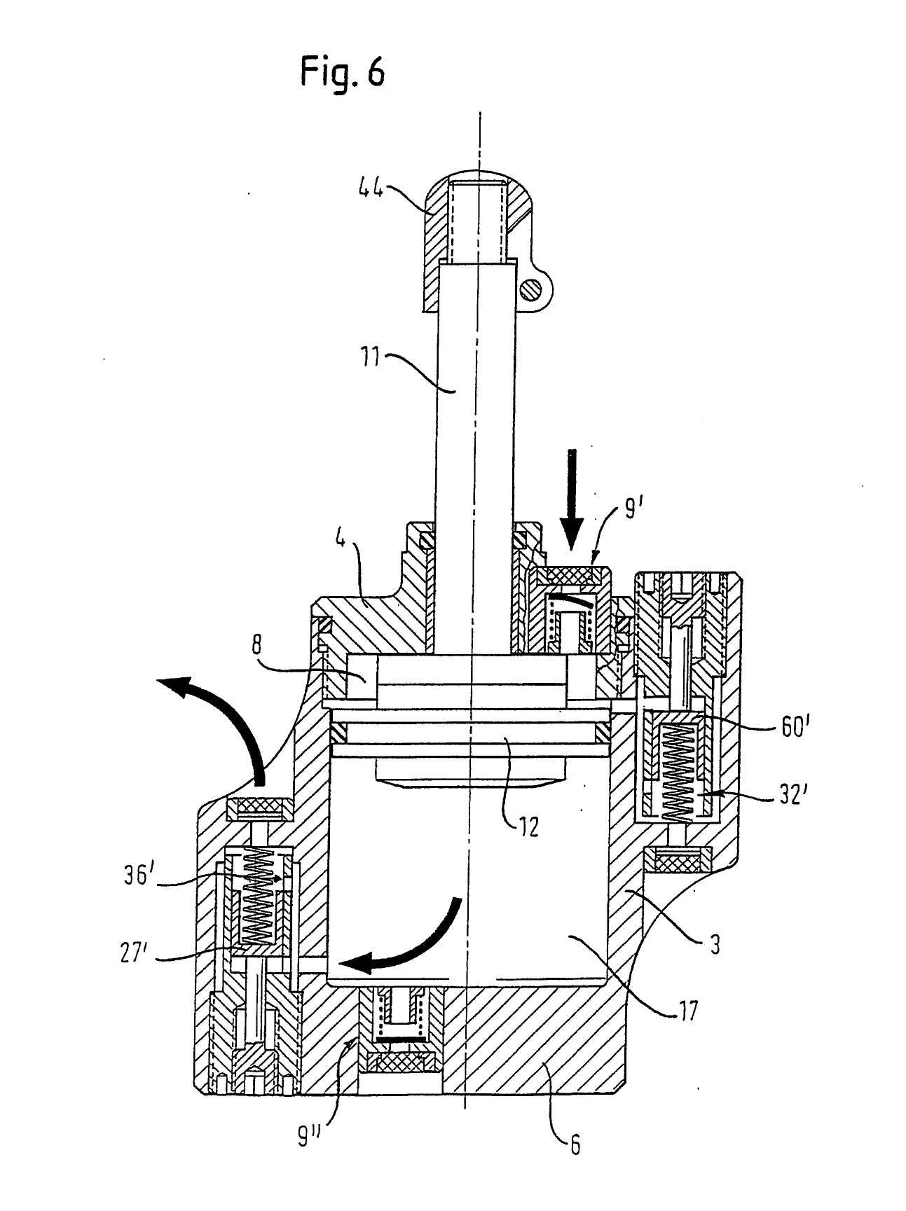

FIG. 6 shows a schematic illustration of a swing phase control device according to a further embodiment of the present invention during a motion of flexion;

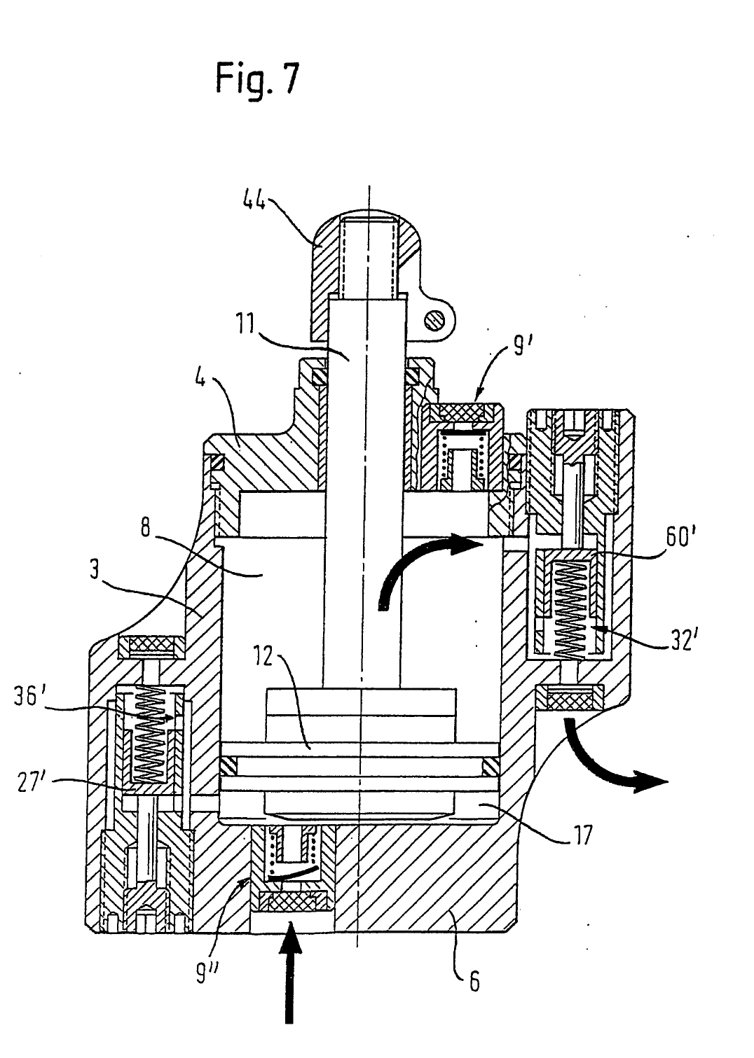

FIG. 7 shows a schematic illustration of the swing phase control device shown in FIG. 6 during a motion of extension.

In the following, a first embodiment of the present invention will be described with reference to FIGS. 1 through 4.

The swing phase control device 1 comprises a cylinder-piston device 2 with a cylinder 3 and a piston 12. The cylinder 3 comprises a tube-shaped portion 5 with a cap part 4 on its one end and a bottom part 6 on the opposite end.

The cap part 4 comprises an inlet opening 7 which allows connection between the ambient air and an input-side first chamber 8 formed in the cylinder 3. The inlet opening 7 is provided with a return valve 9 which opens towards the first chamber 8 and which, for example, may be formed as a flatter valve. Concentrically to the cylinder 3, a bore 10 is provided in a sleeve in the cap part 4. A slide bearing for a piston rod 11 connected to the piston 12 of the piston-cylinder device 2 is located in the bore 10. A filter 13 for protection against entering of undesired components from the ambient air and for damping noises produced by air passing-through is provided on the outer side of the inlet opening 7. A ring sealing 14 is provided for sealing the space between the concentric bore 10 and the piston rod 11.

The cap part 4 is connected to the tube-shaped portion 5 and the connection between the tube-shaped portion 5 and the cap part 4 is sealed by a ring sealing 15, for example. The bottom part 6 closes the tube-shaped portion 5 at its end opposite to the cap part 4 and is formed in one piece with the tube-shaped portion 5. The bottom part 6 can also be separately formed and can be screwed together or otherwise connected to the tube-shaped portion 5.

On the side of the piston 12 facing away from the first chamber 8, a second chamber 17 is formed in the tube-shaped portion 5. On the side opposite to the piston 12, the second chamber 17 is delimited by the bottom part 6.

A bore 18 opening towards the second chamber 17 is concentrically provided in the bottom part 6. The bottom part 6 comprises a radial bore 50 which connects the outer side of the cylinder 3 to the bore 18 and which is provided with a closure element 51. Continuous with the bore 50 towards the opposite outer side of the cylinder 3, a radial outlet bore 19 is provided which connects the outer side of the cylinder 3, and thus the ambient air, to the bore 18. The portion 19a of the outlet bore 19 which faces the outer side is provided with a filter 20 for preventing dust from the ambient air from entering and for damping noises arising due to the air passing through the outlet bore 19. In the portion 19b, which adjoins the portion 19a in the direction of the bore 18, the radial outlet bore 19 comprises a tapered cross-section and in the portion l9c directly adjoining the bore 50 a larger cross-section again which is slightly smaller than the cross-section of the bore 50.

The passage from the bore 18 to the portion 19c is partly blocked by the closure element 51 such that a throttle 32 is formed which throttles flowing-out of air from the second chamber 17 through the bores 18, 50 and 19. The closure element 51 is formed such that it blocks the region of the bore 50 facing away from the bore 19 and protrudes into the passage from the bore 18 to the portion 19c with a protrusion 51a shaped as a hollow cylinder.

A control element 27 which is coaxially displaceable in the closure element 51 is accommodated in the hollow-cylindrical inner space of the closure element 51. The control element 27 is formed as cup-shaped and the bottom face 27a of the cup forms a side wall of a space 51c in the hollow-cylindrical inner space of the closure element 51. This space 51c communicates with the bore 18 via a lateral bore 51b in the hollow-cylindrical protrusion 51a. In a first end position, this control element 27 completely blocks the throttle 32 and, in a direction towards a second end position, it successively unblocks the throttle 32. This control element 27 is pre-biased into the second end position against an abutment 25 protruding into the space 51c by a pressure spring 30 reaching into the inner space of the cup-shaped control element 27. The position of the abutment 25 can be adjusted by an adjustment screw 23 accessible from the outside.

When air flows from the second chamber 17 through the bore 18 and into the bore 50, it presses against the control element 27 on the side facing away from the pressure spring 30 and, when exceeding the spring force, moves the control element 27 in the direction of the position blocking the throttle 32.

The piston 12 further comprises a concentric bore 35 which opens towards the second chamber 17. On a side facing away from the second chamber 17, the concentric bore 35 communicates with the first chamber via a transverse bore 36. The concentric bore 35 comprises a return valve 37 opening towards the second chamber which again can be formed as a flatter valve, for example. The piston 12 comprises a ring sealing 39 resting against the tube-shaped portion 5. The concentric bore 35 of the piston 12 continuously extends in the piston rod 11 which is connected to the piston 12 at the side facing the cap part 4. Thus, the piston rod 11 is formed as hollow with a cylindrical cross-section in a portion adjoining the concentric bore 35.

A second control element 60 is arranged in the piston rod 11 coaxially to the latter. The second control element 60 comprises an outer diameter which is selected corresponding to the inner diameter of the hollow piston rod 11 such that the second control element 60 fills the cross-section of the piston rod 11 and is displaceable therein along the rod axis. The second control element 60 is arranged in the piston rod 11 such that, in a first end position, it completely blocks the transverse bore 36 which forms a throttle for air flowing from the first chamber 8 in the direction to the second chamber 17 and, with movement in the direction towards a second end position, successively unblocks the transverse bore 36.

On the side facing the piston 12 of the second control element 60, a second pressure spring 61 is provided in the piston rod 11 pre-biasing the second control element 60 in the direction towards the second end position against an abutment 62 provided in the piston rod 11.

In the piston rod 11 on the side facing away from the piston 12 of the second control element 60, a second transverse bore 63 is provided connecting the inner space of the hollow piston rod 11 to the first chamber 8. In the direction of the free end 11a of the piston rod 11 the inner space of the hollow piston rod 11 is delimited by a second closure element 64. The pressure in the first chamber 8 acts on the upper side 60a facing away from the second pressure spring 61 of the second control element 60 via the second transverse bore 63. The second closure element 64 can be adjusted in its position in the direction of the piston rod axis via a screw accessible from the free end 11a such that the transverse bore 36 can partly be pre-blocked with the control element 60 via the abutment 62 against the pressure of the second pressure spring 61. With this arrangement an adjustable throttle is formed the cross-section of which becomes reduced dependent on the pressure in the first chamber and thus dependent on the speed of extension of the piston from the cylinder.

In use in an artificial knee joint, shown in FIG. 5, the swing phase control device 1 is connected to the prosthesis thigh part via a fastening part 44 connected to the free end 11a of the piston rod 11. The cylinder 3 is connected to the prosthesis lower part in its bottom region via recesses 45 formed therein.

In the following, operation of the swing phase control device will be described. When the artificial knee joint is extended (extension), the piston 12 is in its retracted upper position adjacent to the cap part 4. The first chamber 8 has its minimum size. This situation is shown in FIGS. 1, 3 and 4, respectively. The second chamber 17 has its maximum size. If the artificial leg of the person using the swing phase control device becomes bent, the knee joint becomes bent (flexion). The piston 12 moves from its upper position into the lower position and ambient air flows through the return valve 9 into the enlarging first chamber 8. The pressure spring 30 and the diameter of the throttle 32 are dimensioned such that, under normal operation, the pressure in the second chamber 17 is not capable of pressing the control element 27 in a position closing the throttle 32. Thus, the throttle 32 is unblocked and the piston 12 moving downwards displaces the air present in the second chamber 17 through the bores 18, 50 and 19 and to the outside.

If, however, the person wearing the prosthesis moves fast and, in doing so, bents the knee joint, the piston 12 rapidly moves into the lower position. Thereby, a high pressure rapidly develops in the second chamber 17 since, due to the throttle 32, the air cannot flow fast enough from the second chamber 17 to the outlet bore 19 via the bore 18 and the throttle 32. If the bending motion continues long enough, the pressure in the second chamber 17 rises high enough for the pressure acting on the control element 27 to become sufficient to move the control element 27 against the action of the pressure spring 30 into a position slightly closing the throttle 32. Due to the blocking of the throttle 32, the pressure in the second chamber 17 rises even faster and the control element 27 blocks the throttle 32 even faster such that, finally, it is completely blocked. Then, the second chamber acts as an air spring, since the air only becomes compressed and cannot flow out any longer.

The air spring formed by the second chamber 17 effects that the retracting motion of the piston 12 in the cylinder 3 becomes reversed and the piston 12 is extended from the cylinder 3 again. Since the upward motion of the piston 12 means extension of the knee joint, the motion of extension of the knee joint is actively supported by reversal of the motion of the piston 12 caused by the air spring in the second chamber 17.

In the motion of extension of the knee joint, when the piston 12 becomes moved out from the cylinder 3, air from the first chamber 8 flows into the enlarging second chamber 17 via the transverse bore 36, the bore 35 and the return valve 37 permeable in this direction. The second pressure spring 61 and the diameter of the transverse bore 36 are dimensioned such that, under normal operation, the pressure in the first chamber 8 is not capable of pressing the second control element 60 in a position blocking the transverse bore 36. As a result, the transverse bore 36 is unblocked and the piston 12 moving upwards displaces the air present in the first chamber 8 into the second chamber 17.

However, if the person wearing the prosthesis moves fast again and, in doing so, extends the knee joint, the piston 12 rapidly moves in the upper position. Thus, a high pressure rapidly arises in the first chamber 8 since, due to the transverse bore 36, the air cannot flow fast enough into the second chamber 17 through the transverse bore 36 and the bore 35. If the motion of extension continues long enough, the pressure in the first chamber 8 increases high enough for the pressure acting on the second control element 60 to become sufficient to move the second control element 60 against the action of the second pressure spring 61 into a position slightly blocking the transverse bore 36. Due to the blocking of the transverse bore 36, the pressure in the first chamber 8 rises even faster and the second control element 60 blocks the transverse bore 36 even faster such that, finally, it is completely blocked. Then, the first chamber acts as an air spring.

The air spring formed by the first chamber 8 effects that the extending motion of the piston 12 from the cylinder 3 becomes reversed and that the piston 12 becomes retracted into the cylinder 3 again. Since the downward motion of the piston 12 means bending of the knee joint, the bending motion of the knee joint is also actively supported by the reversal of the motion of the piston 12 caused by the air spring in the first chamber 8.

Thus, the motion of bending and the motion of extension of the knee joint are actively supported depending on the speed of motion such that the person can move much faster and more save. The timings of the respective reverse motions caused by the air springs can be adapted to the individual circumstances by adjusting the respective throttling effects by adjusting the abutments 25, 62.

In the above described embodiment the first chamber 8 and the second chamber 17 are connected to the ambient air via the inlet opening 7 and the outlet bore 19, respectively. Thus, an open system is provided in which air is used as a medium. However, a closed system can be provided as well in which the swing phase control is surrounded by a hermetically sealed outer casing. In the case of such an alternative embodiment also another gas or a liquid can be used as moved medium. With such an embodiment the characteristic curve of the swing phase control can be influenced. However, using a liquid, due to the low compressibility, does not provide the back-cushioning property which has been described above.

In a further embodiment it is also possible to reverse the throttle device 36, 63, 60, 61 provided in the piston 12 and in the piston rod 11, such that it serves as a throttle device for a fluid flowing from the second chamber 17 into the first chamber 8, and to provide the other throttle device 18, 19, 32, 30, 27 in the first chamber 8 between the first chamber and the outer side of the cylinder 3, such that it serves as a throttle device for a fluid flowing from the first chamber 8 to the outside. In this case, the inlet opening 7 with return valve 9 is arranged in the second chamber 17.

In a further embodiment shown in FIGS. 6 and 7 none of the throttles is provided in the piston 12 but both throttles 32′, 36′ are each formed in a passage to the ambient air. In each of the chambers 8, 17 a return valve 9′, 9″ is provided which connects the respective chamber 8, 17 to the outside of the cylinder 3 and opens towards the chamber 8, 17.

In FIG. 6 the flow paths of the fluid during a motion of flexion of the artificial knee joint are schematically illustrated by arrows. During the motion of flexion, as in the previously described embodiment, the piston 12 moves downwards and compresses the fluid in the second chamber 17. During this, the return valve 9″ at the second chamber blocks such that it does not form a passage for the fluid from the second chamber 17 to the outside of the cylinder 3. As a result, the fluid flows from the second chamber 17 to the outside of the cylinder 3 via the throttle 36′. The fluid flows from the outside of the cylinder 3 into the first chamber 8 through the opened return valve 9′. As in the first embodiment, the throttle 36′ is provided with a control element 27′. which blocks the throttle 36′ in case of a high pressure in the second chamber 17.

During the motion of extension illustrated in FIG. 7 the piston 12 moves upwards and the fluid in the first chamber 8 becomes compressed. Fluid from the outside of the cylinder 3 flows into the second. chamber 17 through the return valve 9″, at the second chamber 7 such that no negative pressure arises in the latter. The return valve 9′ at the first chamber 8 prevents direct flowing-out of the fluid from the first chamber 8 such that it flows from the first chamber 8 to the outside of the cylinder via the throttle 32′. Like the throttle 36′, the throttle 32′ at the first chamber 8 is provided with a control element 60′ closing the throttle 32′ in the case of high pressure arising in the first chamber 8.

As a further alternative, it is conceivable to arrange both throttles in the piston 12 or in the piston 12 and in the piston rod 11 and to arrange the throttles in series with return valves, respectively, in two channels. With such a construction, a closed system using another fluid different from ambient air can be realized.

1.-11. (canceled)

12. swing phase control device for an artificial knee joint, comprising:
a piston-cylinder device, comprising:
a piston having a first side and a second side opposite the first side;
a first chamber associated with the first side of the piston; and
a second chamber associated with the second side of the piston;
a first throttle for throttling discharge of a fluid from the first chamber and configured to be closed by a first device when a predetermined pressure is reached or exceeded within the first chamber; and
a second throttle for throttling discharge of a fluid from the second chamber and configured to be closed by a second device when a predetermined pressure is reached or exceeded within the second chamber.

13. The swing phase control device according to claim 12, wherein the first throttle is disposed within the piston.

14. The swing phase control device according to claim 12, wherein the first chamber and the second chamber may be in fluid connection via the first throttle.

15. The swing phase control device according to claim 12, wherein the first chamber is connectable to a fluid reservoir.

16. The swing phase control device according to claim 12, wherein the second throttle is disposed on an output side of the second chamber and facing away from the piston.

17. The swing phase control device according to claim 12, wherein the second chamber is connectable to a fluid reservoir via the second throttle.

18. The swing phase control device according to claim 12, wherein the fluid is air.

19. The swing phase control device according to claim 12, wherein the fluid reservoir is formed by the ambient air.

20. The swing phase control device according to claim 12, wherein the fluid is a hydraulics medium and the fluid reservoir is pressure compensated with the surrounding area.

21. The swing phase control device according to claim 12, wherein the fluid is a pneumatics medium and the fluid reservoir is pressure compensated with the surrounding area.

22. The swing phase control device according to claim 12, wherein the first device comprises:
a control piston movable to and fro along a path.

23. The swing phase control device according to claim 12, wherein the second device comprises:
a control piston movable to and fro along a path.

24. The swing phase control device according to claim 12, wherein the first device comprises:
an element configured to apply a restoring force which unblocks the first throttle when the pressure within the first chamber falls below the predetermined pressure.

25. The swing phase control device according to claim 12, wherein the second device comprises:
an element configured to apply a restoring force which unblocks the second throttle when the pressure within the second chamber falls below the predetermined pressure.
